(19) United States
(12) Patent Application Publication
(10) Pub. No.: US 2026/0090978 A1
(43) Pub. Date: Apr. 2, 2026

(54) MASCARA COMPOSITION

(71) Applicant: L’ORÉAL, Paris (FR)

(72) Inventors: David CHAN, Oradell, NJ (US); Geoffrey WHITE, Old Bridge, NJ (US)

(73) Assignee: L’ORÉAL, Paris (FR)

(21) Appl. No.: 18/898,887

(22) Filed: Sep. 27, 2024

(51) Int. Cl. A61K 8/92; A61Q 1/10

(52) U.S. Cl. CPC A61K 8/922; A61Q 1/10; A61K 2800/30; A61K 2800/43; A61K 2800/5922

(57) ABSTRACT

Mascara compositions with improved aesthetics are provided. Such compositions generally include a plurality of waxes including sunflower seed wax and a plurality of behenates including tribehenin and behenyl behenate. The plurality of waxes may include the sunflower seed wax, the tribehenin, the behenyl behenate, and at least one additional wax. The plurality of waxes may be present in a total amount of 5% to 35% by weight of the mascara composition. The combined total amount of sunflower seed wax, tribehenin, and behenyl behenate may be 5% to 20% by weight of the composition. The mascara composition may optionally include no more than 10% water, such as 2%-7%, by weight of the mascara composition.

### TECHNICAL FIELD

The present disclosure is drawn to cosmetic compositions, such as mascara compositions.

### BACKGROUND

Cosmetic products, such as washable versions of mascaras, are used to provide clean, regular end looks for a user. However, such products tend to smear, run, or otherwise have low resistance to, e.g., water. Conventional techniques for improving water resistance tends to result in mascaras that are anhydrous and include high levels of waxes, and volatile solvents. Due to the high level of waxes and volatile solvents, common complaints of waterproof mascara users are that such products provide a makeup effect that is very clumpy and deposit visible grains of product onto lashes. The result is a consumer with a messy and irregular end look that is undesirable.

### BRIEF SUMMARY

In certain aspects, a mascara composition may be provided. The mascara composition may include one or more waxes, and may optionally include water. The one or more waxes include sunflower seed wax and a plurality of behenates. The plurality of behenates include at least tribehenin and behenyl behenate. Water, if present, may be present in an amount no more than 10%, such as 2%-7%, by weight of the mascara composition.

The waxes may be present in a total amount of 5%-35% by weight of the mascara composition. In some embodiments, the sunflower seed wax, the tribehenin, and the behenyl behenate are present in a total amount of 5% to 20% by weight of the composition. In certain aspects, the one or more waxes may include the sunflower seed wax, the tribehenin, the behenyl behenate, and at least one additional wax. One or more additional wax(es) may have melting temperatures at or below 60° C. One or more additional wax(es) may have melting temperatures above 60° C.

The mascara may include one or more additional components. Such components may include, e.g., a colorant, which may be a pigment, or a volatile solvent. The composition may be substantially free of a non-volatile solvent.

In certain aspects, a method for providing improved mascara aesthetics may be provided. The method may include applying a mascara composition as disclosed herein to one or more lashes. The method may include allowing the mascara composition to remain on the one or more lashes for a period of time of, e.g., 1 hour to 48 hours.

### DETAILED DESCRIPTION

As used herein, the terms “a,” “an,” and “the” are understood to encompass the plural as well as the singular.

The formulation of environmentally friendly cosmetic products, which are designed and developed considering environmental issues, is becoming a major goal in an effort to meet global challenges. It is therefore essential to propose more sustainable compositions, preparation processes and ingredients to address these environmental concerns.

In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin.

Disclosed are mascara compositions configured to deliver volume to lashes in low water/anhydrous mascara compositions, while minimizing the graininess of the deposition through the combination of sunflower seed wax, tribehenin and behenyl behenate.

Specifically, the disclosed mascara compositions may include a plurality of specific waxes. As used herein, the term “wax” generally refers to a lipophilic compound that is solid at room temperature (25° C.), with a solid/liquid reversible change of state, having a melting point of greater than or equal to 30° C., which may be up to 200° C. and in particular up to 120° C.

The waxes include sunflower seed wax and a plurality of behenates. The plurality of behenates include at least tribehenin and behenyl behenate. In one preferred embodiment, the plurality of behenates includes only tribehenin and behenyl behenate. In some embodiments, the plurality of behenates may include tribehenin, behenyl behenate, and one or more additional behenates, such as, e.g., glyceryl behenate.

The plurality of waxes may be present in a total amount of at least 1%, at least 2%, at least 3%, at least 4%, at least 5%, at least 6%, at least 7%, at least 8%, at least 9%, at least 10%, at least 11%, at least 12%, at least 13%, or at least 14% up to no more than 15%, no more than 16%, no more than 17%, no more than 18%, no more than 19%, no more than 20%, no more than 25%, no more than 30%, or no more than 35% by weight of the mascara composition, including all ranges and subranges there between.

In some embodiments, the sunflower seed wax, the tribehenin, and the behenyl behenate may be present in a total amount of at least 5%, at least 6%, at least 7%, at least 8%, at least 9%, or at least 10%, up to no more than 12%, no more than 13%, no more than 14%, no more than 15%, no more than 16%, no more than 17%, no more than 18%, no more than 19%, or no more than 20% by weight of the composition, including all ranges and subranges there between.

The sunflower seed wax may be present in a total amount of at least 1%, at least 2%, at least 3%, at least 4%, or at least 5%, up to no more than 6%, no more than 7%, no more than 8%, no more than 9%, no more than 10%, no more than 11%, no more than 12%, no more than 13%, or no more than 15% by weight of the mascara composition, including all ranges and subranges there between. In preferred embodiments, the sunflower seed wax is present in an amount of 5%-10% by weight of the composition.

The tribehenin may be present in a total amount of at least 0.1%, at least 0.5%, at least 1%, at least 2%, at least 3%, at least 4%, at least 5%, at least 5.1%, at least 5.2%, at least 5.3%, a least 5.4%, or at least 5.5%, up to no more than 6%, no more than 7%, no more than 8%, no more than 9%, no more than 10%, no more than 11%, no more than 12%, no more than 13%, or no more than 15% by weight of the mascara composition, including all ranges and subranges there between. In preferred embodiments, the tribehenin is present in an amount of 1%-7% by weight of the composition.

The behenyl behenate may be present in a total amount of at least 0.1%, at least 0.5%, at least 1%, at least 1.5%, or at least 2%, up to no more than 3%, no more than 4%, no more than 5%, no more than 6%, no more than 7%, no more than 8%, no more than 9%, no more than 10%, no more than 11%, no more than 12%, no more than 13%, or no more than 15% by weight of the mascara composition, including all ranges and subranges there between. In preferred embodiments, the behenyl behenate is present in an amount of 2%-6% by weight of the composition.

In some embodiments, the amount of sunflower seed wax plus the amount of behenyl behenate may be at least 7.1%, at least 7.2%, at least 7.3%, at least 7.4%, or at least 7.5%, up to no more than 8%, no more than 9%, no more than 10%, no more than 11%, no more than 12%, no more than 15%, no more than 20%, no more than 25%, or no more than 30% by weight of the mascara composition, including all ranges and subranges there between.

### Additional Waxes

The plurality of waxes may include one or more additional waxes. That is, in some embodiments, the plurality of waxes may include only the sunflower seed wax, the tribehenin, and the behenyl behenate. In some embodiments, the plurality of waxes may include the sunflower seed wax, the tribehenin, and the behenyl behenate, and at least one additional wax.

The at least one additional wax may include a soft wax. As used herein, the term “soft wax” refers to waxes which have a melting point of no more than about 70° C., and preferably, a melting point of no more than about 60° C.

As used herein, the term “about [a number]” generally refers to values, as appropriate, that are within +10%, +8%, +6%, +5%, +4%, +3%, +2%, +1%, or +0.5% of the number, or just including the number itself.

The at least one additional wax may be substantially free of a soft wax. The at least one additional wax may be free of a soft wax.

As used herein, the term “free” or “completely free (of a component)” refers to compositions that do not contain the component in any measurable degree by standard means. As used herein, the term “substantially free (of a component)” refers to compositions that contain no appreciable amount of the component, for example, no more than about 1% by weight, or no more than about 0.5% by weight, or no more than about 0.3% by weight, such as no more than about 0.1% by weight, based on the weight of the composition.

Non-limiting examples of soft waxes include paraffin wax, ozokerite, synthetic beeswax, beeswax, candelilla wax, hydrogenated jojoba wax, palm butter, sumac wax, polyglyceryl beeswax, siliconyl beeswax, ceresin wax, orange peel wax, silicone resin wax, alkyl dimethicone wax, Berry wax, koster wax, siliconyl candelilla wax, and montan wax.

The at least one additional wax may include a hard wax. As used herein, the term “hard wax” refers to waxes other than soft waxes—that is, waxes which have a melting point greater than about 70° C., and preferably, a melting point greater than about 60° C. The at least one additional wax may be substantially free of a hard wax. The at least one additional wax may be free of a hard wax.

Non-limiting examples of hard waxes include carnauba wax, microcrystalline wax, polyethylene wax, hydrogenated castor oil, wax AC 540, hydrogenated castor wax, wax AC 400, rice bran wax, Alcohol polyethylene wax, sunflower seed wax, fischer-tropsch wax, Chinese insect wax, and shellac wax.

In some embodiments, the additional waxes may include at least one hard wax and at least one soft wax. In some embodiments, the additional waxes may include one hard wax and one soft wax.

The additional waxes may be present in a total amount of at least 0.5%, at least 1%, at least 1.5%, or at least 2%, up to no more than 4%, no more than 5%, no more than 6%, no more than 7%, no more than 8%, no more than 9%, no more than 10%, no more than 11%, no more than 12%, no more than 13%, no more than 14%, or no more than 15% by weight of the mascara composition, including all ranges and subranges there between. In preferred embodiments, the additional waxes are present in an amount of 2%-4% by weight of the composition.

In some embodiments, the amount of sunflower seed wax plus the amount of behenyl behenate and any additional wax(es) may be at least 7.1%, at least 7.2%, at least 7.3%, at least 7.4%, or at least 7.5%, up to no more than 12%, no more than 13%, no more than 14%, no more than 15%, no more than 20%, no more than 25%, no more than 30%, or no more than 35% by weight of the mascara composition, including all ranges and subranges there between.

The composition may include additional components.

### Volatile Solvents

The additional components may include a volatile solvent. The term “volatile solvent” means a solvent that is capable of evaporating on contact with keratin materials in less than one hour, at room temperature and atmospheric pressure (760 mmHg). Such volatile solvents are typically liquid at room temperature, with a non-zero vapor pressure at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg). By way of example, the vapor pressure may be measured according to the static method or via the effusion method by isothermal gravimetry, depending on the vapor pressure (standard OCDE 104).

The mascara composition may include volatile solvents in a total amount of at least 1%, at least 5%, at least 10%, at least 15%, at least 20%, at least 25%, at least 30%, at least 35%, at least 40%, at least 45%, or at least 50%, up to no more than 55%, no more than 60%, no more than 65%, no more than 70% by weight of the composition, including all ranges and subranges there between.

### Volatile Non-Oil Solvents

The volatile solvent may be a non-oil liquid volatile solvent. The term “oil” is intended to mean a non-aqueous compound, non-miscible in water, liquid, at 25° C. and atmospheric pressure (760 mmHg; 1.013.10⁵ Pa). The term “non-oil” refers to all other materials other than oils.

Such non-oil solvents are typically water, or water-miscible materials. Non-limiting examples of such solvents include a polar protic solvent, such as water and C2-C5 monoalcohols such as ethanol, isopropanol, butyl alcohol, etc.

The mascara composition may include a polar protic solvent in a total amount of at least 1%, at least 1.5%, at least 2%, at least 2.5%, at least 3%, at least 3.5%, at least 4%, at least 4.5%, at least 5%, up to no more than 5.5%, no more than 6%, no more than 6.5%, no more than 7%, no more than 7.5%, or no more than 8% by weight of the composition, including all ranges and subranges there between. In a preferred embodiment, a polar protic solvent is present in a total amount of 4% to 7%. In some embodiments, the composition may be substantially free of a polar protic solvent. In some embodiments, the composition may be free of a polar protic solvent.

The mascara composition may include water in an amount of at least 1%, at least 1.5%, at least 2%, at least 2.5%, at least 3%, or at least 3.5%, up to no more than 4%, no more than 4.5%, no more than 5%, no more than 5.5%, no more than 6%, no more than 6.5%, no more than 7%, no more than 8%, no more than 9%, or no more than 10% by weight of the composition, including all ranges and subranges there between. In a preferred embodiment, water is present in an amount of 1% to 8%. In a more preferred embodiment, water is present in an amount of 2% to 7, and in a still more preferred embodiment, water is present in an amount of 3% to 5% by weight of the composition. In some embodiments, the composition may be substantially free of water. In some embodiments, the composition may be free of water.

The mascara composition may include C2-C5 monoalcohol(s) in a total amount of at least 0.5%, at least 1%, or at least 1.5%, up to no more than 2%, no more than 2.5%, no more than 3%, no more than 3.5%, no more than 4%, no more than 4.5%, or no more than 5%, including all ranges and subranges there between. In a preferred embodiment, is C2-C5 monoalcohol is present in an amount of 1% to 3%. In some embodiments, the composition may be substantially free of a C2-C5 monoalcohol. In some embodiments, the composition may be free of C2-C5 monoalcohol.

The non-oil solvent may include an aprotic solvent, such as a polar aprotic solvent. Such polar aprotic solvents include, e.g., propylene carbonate, dimethyl sulfoxide (DMSO), etc.

The mascara composition may include a polar aprotic solvent in a total amount of at least 0.5%, at least 1%, or at least 1.5%, up to no more than 2%, no more than 2.5%, no more than 3%, no more than 3.5%, no more than 4%, no more than 4.5%, or no more than 5%, by weight of the composition, including all ranges and subranges there between. In a preferred embodiment, a polar protic solvent is present in a total amount of 1% to 4%. In some embodiments, the composition may be substantially free of a polar aprotic solvent. In some embodiments, the composition may be free of a polar aprotic solvent.

In certain embodiments, the non-oil solvent may include only water. In certain embodiments, the non-oil solvent may include only water and a single C2-C5 monoalcohol. In certain embodiments, the non-oil solvent may include only water, a single C2-C5 monoalcohol, and a single polar aprotic solvent.

### Volatile Oil Solvents

The volatile solvent may include a volatile oil solvent. The volatile oil solvent may be a silicone oil. The term “silicone oil” means an oil comprising at least one silicon atom, and notably at least one Si—O group, and more particularly an organopolysiloxane.

The mascara composition may include volatile oil solvents in a total amount of at least 1%, at least 5%, at least 10%, at least 15%, at least 20%, at least 25%, at least 30%, at least 35%, at least 40%, at least 45%, or at least 50%, up to no more than 55%, no more than 60%, no more than 65%, no more than 70% by weight of the composition, including all ranges and subranges there between.

### Volatile Silicone Oils

The volatile solvents may include a volatile silicone oil. Non-limiting examples of volatile silicone oils include volatile linear or cyclic silicone oils, especially those with a viscosity≤8 centistokes (cSt) (8×10⁻⁶ m²/s), and especially containing from 2 to 10 silicon atoms and in particular from 2 to 7 silicon atoms, these silicones optionally comprising alkyl or alkoxy groups containing from 1 to 10 carbon atoms. As volatile silicone oils that may be used in the invention, mention may be made especially of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyl-octyltrisiloxane, hexamethyldisiloxane, octamethyltrisiloxane, decamethyltetrasiloxane and dodecamethylpentasiloxane, and mixtures thereof.

The mascara composition may include volatile silicone oils in a total amount of at least 1%, at least 5%, at least 10%, at least 15%, at least 20%, at least 25%, at least 30%, at least 35%, or at least 40%, up to no more than 50%, no more than 55%, no more than 60%, no more than 65%, no more than 70% by weight of the composition, including all ranges and subranges there between. In some embodiments, the composition may be substantially free of volatile silicone oils. In a preferred embodiment, the composition may be free of volatile silicone oils.

### Non-Volatile Solvents

The additional components may include a non-volatile solvent. The term “non-volatile solvent” refers to a solvent that remains on the skin or a keratin fiber at room temperature and atmospheric pressure for at least several hours, and that notably has a vapor pressure of less than 2.66 Pa, preferably less than 0.13 Pa.

The non-volatile solvent may be a non-volatile oil. The non-volatile oil may be a non-volatile hydrocarbon-based oil, or a non-volatile silicone oil. The term “hydrocarbon-based oil” refers to an oil mainly containing carbon and hydrogen atoms and possibly one or more functions chosen from hydroxyl, ester, ether and carboxylic functions.

The mascara composition may include non-volatile solvents in a total amount of at least 1%, at least 5%, or at least 10%, up to at no more than 20%, no more than 30%, no more than 40%, or no more than 50%, including all ranges and subranges there between. In a preferred embodiment, the mascara composition may be substantially free of a non-volatile solvent. In a more preferred embodiment, the mascara composition may be free of a non-volatile solvent.

### Non-Volatile Hydrocarbon-Based Oils

Non-limiting examples of non-volatile hydrocarbon-based oils include:
hydrocarbon-based oils of animal origin, such as perhydrosqualene;
linear or branched hydrocarbons, of mineral or synthetic origin, such as liquid paraffins and derivatives thereof, petroleum jelly, polydecenes, polybutenes or polyisobutenes, which are optionally hydrogenated such as Parleam, or squalane;
synthetic ethers containing from 10 to 40 carbon atoms, such as dicaprylyl ether;
triglycerides consisting of fatty acid esters of glycerol, in particular the fatty acids of which may have chain lengths ranging from C4 to C36, and notably from C18 to C36, these oils possibly being linear or branched, and saturated or unsaturated; these oils may notably be heptanoic or octanoic triglycerides, wheatgerm oil, sunflower oil, grapeseed oil, sesame seed oil (820.6 g/mol), corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil or musk rose oil; shea oil; or alternatively caprylic/capric acid triglycerides;
linear aliphatic hydrocarbon-based esters of formula RCOOR′ in which RCOO represents a carboxylic acid residue including from 2 to 40 carbon atoms, and R′ represents a hydrocarbon-based chain containing from 1 to 40 carbon atoms, such as cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, diisopropyl adipate, heptanoates, and notably isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, hexyl laurate, neopentanoic acid esters, for instance isodecyl neopentanoate, isotridecyl neopentanoate, isostearyl neopentanoate and 2-octyldodecyl neopentanoate, isononanoic acid esters, for instance isononyl isononanoate, isotridecyl isononanoate and octyl isononanoate, oleyl erucate, isopropyl lauroyl sarcosinate, diisopropyl sebacate, isocetyl stearate, isodecyl neopentanoate and isostearyl behenate;
polyesters obtained by condensation of unsaturated fatty acid dimer and/or trimer and of diol, in particular such as of dilinoleic acid and of 1,4-butanediol. In some embodiments, this may include, e.g., dilinoleic acid/butanediol copolymer or copolymers of polyols and of diacid dimers, and esters thereof;
dialkyl carbonates, the two alkyl chains possibly being identical or different, such as dicaprylyl carbonate;
linear fatty acid esters with a total carbon number ranging from 35 to 70, for instance pentaerythrityl tetrapelargonate,
aromatic esters such as tridecyl trimellitate C12-C15 alcohol benzoate, the 2-phenylethyl ester of benzoic acid, and butyloctyl salicylate,
esters and polyesters of diol dimer and of monocarboxylic or dicarboxylic acid, such as esters of diol dimer and of fatty acid and esters of diol dimer and of dicarboxylic acid dimer, such as Lusplan™ DD-DA5 oligomeric polyester oil and Lusplan™ DD-DA7 ester sold by the company Nippon Fine Chemical,
fatty alcohols containing from 12 to 26 carbon atoms, for instance octyldodecanol, 2-butyloctanol, 2-hexyldecanol, 2-undecylpentadecanol and oleyl alcohol;
and mixtures thereof.

In some embodiments, the mascara composition may be substantially free of non-volatile hydrocarbon-based oils. In some embodiments, the mascara composition may be free of non-volatile hydrocarbon-based oils.

In some embodiments, the mascara composition may be substantially free of non-volatile hydrocarbon-based oils. In some embodiments, the mascara composition may be free of non-volatile hydrocarbon-based oils.

### Non-Volatile Silicone Oils

Non-limiting examples of non-volatile silicone oils include non-volatile polydimethylsiloxanes (PDMS); phenyl silicones such as phenyl trimethicones, phenyl dimethicones, diphenyl dimethicones, trimethylpentaphenyltrisiloxanes, tetramethyltetraphenyltrisiloxanes, trimethylsiloxyphenyl dimethicones, diphenylsiloxyphenyl trimethicones, and also mixtures thereof.

In some embodiments, the mascara composition may be substantially free of non-volatile silicone oils. In some embodiments, the mascara composition may be free of non-volatile silicone oils.

### Colorant

The mascara composition may include a colorant. The colorant may be, e.g., a pigment.

### Pigment

In certain aspects, the composition may include a pigment.

Examples of suitable pigments include, but are not limited to, inorganic pigments, organic pigments, lakes, pearlescent pigments, iridescent or optically variable pigments, and mixtures thereof. The pigments may be chosen from the organic and/or mineral pigments known in the art. The pigments may be in the form of powder or of pigmentary paste. They may be coated or uncoated. Said pigments may optionally be surface treated within the scope of the present invention but are not limited to treatments such as silicones, perfluorinated compounds, lecithin, and amino acids.

The pigments may be chosen, for example, from inorganic pigments, organic pigments, lakes, pigments with special effects such as nacres or glitter flakes, and mixtures thereof. Non-limiting examples of inorganic pigments include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

The pigment may be an inorganic pigment. Non-limiting examples include iron oxides, chromium oxides, manganese violet, ultramarine blue, chromium hydrate, ferric blue and titanium oxide.

The pigment may be an organic pigment. Non-limiting examples include nitroso, nitro, azo, xanthene, quinoline, anthraquinone, phthalocyanin, metal-complex, isoindolinone, isoindoline, quinacridone, perinone, perylene, diketopyrrolopyrrole, thioindigo, dioxazine, triphenylmethane and quinophthalone compounds.

In particular, the white or colored organic pigments may be chosen from carmine, carbon black, aniline black, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and pigments obtained by oxidative polymerization of indole or phenolic derivatives. Non-limiting examples of organic pigments and lakes include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570) and mixtures thereof.

The pigments in accordance with the invention may also be in the form of composite pigments. These composite pigments may be compounds especially of particles comprising a inorganic core, at least one binder for ensuring the binding of the organic pigments to the core, and at least one organic pigment at least partially covering the core.

The organic pigment may also be a lake. The term “lake” refers dyes adsorbed onto insoluble particles, the assembly thus obtained remaining insoluble during use.

The inorganic substrates onto which the dyes are adsorbed are, for example, alumina, silica, calcium sodium borosilicate or calcium aluminum borosilicate, and aluminum.

Among the dyes, mention may be made of cochineal carmine. Non-limiting examples of dyes include: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053), D&C Blue 1 (CI 42 090). An example of a lake is D&C Red 7 (CI 15 850:1).

The pigment may also be a pigment with special effects. The term “pigments with special effects” refers pigments that generally create a non-uniform colored appearance (characterized by a certain shade, a certain vivacity and a certain lightness) that changes as a function of the conditions of observation (light, temperature, observation angles, etc.). They thus contrast with colored pigments that afford a standard uniform opaque, semi-transparent or transparent shade.

Several types of pigment with special effects exist: those with a low refractive index, such as fluorescent, photochromic or thermochromic pigments, and those with a higher refractive index, such as nacres or glitter flakes.

Examples of pigments with special effects that may be mentioned include nacreous pigments such as titanium mica coated with an iron oxide, mica coated with an iron oxide, mica coated with bismuth oxychloride, titanium mica coated with chromium oxide, titanium mica coated with an organic dye especially of the abovementioned type, and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown, gold and/or coppery color or tint.

Non-limiting examples of nacres include gold-colored nacres sold especially by the company Engelhard under the name Gold 222C (a CLOISONNÉ® pigment), Sparkle gold (a TIMICA® pigment), Gold 4504 (a CHROMA-LITE® pigment) and Monarch gold 233X (a CLOISONNÉ® pigment); the bronze nacres sold especially by the company Merck under the name Bronze fine (17384) (a COLORONA® pigment) and Bronze (17353) (a COLORONA® pigment), by the company Eckart under the name PRESTIGE™ Bronze pigment and by the company Engelhard under the name Super bronze (a CLOISONNÉ® pigment); the orange nacres sold especially by the company Engelhard under the name Orange 363C (a CLOISONNE® pigment) and Orange MCR 101 and by the company Merck under the name Passion orange (a COLORONA® pigment) and Matte orange (17449); the brown nacres sold especially by the company Engelhard under the name Nu-antique copper 340XB (a CLOISONNE® pigment) and Brown CL4509 (a CHROMA-LITE® pigment); the nacres with a copper tint sold especially by the company Engelhard under the name Copper 340A (a TIMICA® pigment) and by the company Eckart under the name Prestige Copper; the nacres with a red tint sold especially by the company Merck under the name Sienna fine (17386) (a COLORONA® pigment); the nacres with a yellow tint sold especially by the company Engelhard under the name Yellow (4502) (a CHROMA-LITER pigment); the red nacres with a gold tint sold especially by the company Engelhard under the name Sunstone G012 (a GEMTONE® pigment); the black nacres with a gold tint sold especially by the company Engelhard under the name Nu antique bronze 240 AB (a TIMICA® pigment), the blue nacres sold especially by the company Merck under the name Matte blue (17433), Dark Blue (117324) (a COLORONA® pigment), the white nacres with a silvery tint sold especially by the company Merck under the name XIRONA® Silver pigment, and the golden-green pink-orange nacres sold especially by the company Merck under the name Indian summer (a XIRONA® pigment), and mixtures thereof.

In addition to nacres on a mica support, multilayer pigments based on synthetic substrates such as alumina, silica, sodium calcium borosilicate or calcium aluminum borosilicate, and aluminum, may be envisaged.

Non-limiting examples of pearlescent pigments include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

Mention may also be made of pigments with an interference effect that are not fixed onto a substrate, for instance liquid crystals or holographic interference glitter flakes. Pigments with special effects also comprise fluorescent pigments, whether these are substances that are fluorescent in daylight or that produce an ultraviolet fluorescence, phosphorescent pigments, photochromic pigments, thermochromic pigments and quantum dots.

The variety of pigments that may be used in the present invention makes it possible to obtain a wide range of colors, and also particular optical effects such as metallic effects or interference effects.

The effective diameter of the pigment particles may generally be between 10 nm and 200 μm, such as between 20 nm and 80 μm, such as between 30 nm and 50 μm.

In some embodiments, the pigment may be present in a total amount of no more than 30% by weight of the makeup composition. In some embodiments, the pigment may be present in a total amount of no more than 20% by weight of the makeup composition. The pigment may be present in a total amount of no more than 15% by weight of the makeup composition. The pigment may be present in a total amount of no more than 10% by weight of the makeup composition. The pigment may be present in a total amount of no more than 8% by weight of the makeup composition. The pigment may be present in a total amount of no more than 6% by weight of the makeup composition. The pigment may be present in a total amount of no more than 5% by weight of the makeup composition. The pigment may be present in a total amount of no more than 4% by weight of the makeup composition. The pigment may be present in a total amount of no more than 3% by weight of the makeup composition. The pigment may be present in a total amount of at least 0.1% by weight of the makeup composition. The pigment may be present in a total amount of at least 1% by weight of the makeup composition. The pigment may be present in a total amount of at least 2% by weight of the makeup composition. The pigment may be present in a total amount of at least 3% by weight of the makeup composition. The pigment may be present in a total amount of at least 4% by weight of the makeup composition. The pigment may be present in a total amount of at least 5% by weight of the makeup composition. The pigment may be present in a total amount of at least 10% by weight of the makeup composition. The pigment may be present in a total amount of at least 15% by weight of the makeup composition. The pigment may be present in a total amount of at least 20% by weight of the makeup composition. In some embodiments, the pigment may be present in a total amount of 3-10% by weight of the makeup composition.

In some embodiments, the composition may be substantially free of a pigment. In some embodiments, the composition may be free of a pigment. In some embodiments, the composition may be substantially free of titanium dioxide. In some embodiments, the composition may be free of titanium dioxide.

### Antioxidants

The composition may include an antioxidant. In some embodiments, an antioxidant may be present in the composition in a total amount of 0-0.5% by weight of the composition. In some embodiments, an antioxidant may be present in the composition in a total amount of at least 0.0001%, at least 0.001%, at least 0.01%, or at least 0.1%, up to no more than 0.5%, no more than 1%, no more than 2%, no more than 3%, no more than 4%, or no more than 5% by weight of the composition, including all ranges and subranges thereof. In some embodiments, the composition may be substantially free of an antioxidant. In some embodiments, the composition may be free of an antioxidant.

Non-limiting examples of antioxidants include, e.g., tocopherol, pentaerythrityl tetra-di-t-butyl hydroxyhydrocinnamate, ascorbic acid, ascorbyl compounds such as ascorbyl dipalmitate, t-butylhydroquinone, polyphenols such as phloroglucinol, sodium sulfite, and erythorbic acid.

### Emulsifiers

The composition may include an emulsifier. The emulsifier may be a non-ionic emulsifier.

Non-limiting examples of non-ionic emulsifiers include, e.g., saturated or unsaturated poly(oxyethylenated) C8-C30 monoesters or polyesters; saturated or unsaturated poly(oxyethylenated) C8-C30 alcohols; polyoxyethylenated or non-polyoxyethylenated, saturated or unsaturated C8-C30 monoesters or polyesters of sorbitan; polyoxyethylenated or non-polyoxyethylenated, saturated or unsaturated C8-C30 monoethers or polyethers of sorbitan; alkyl and polyalkyl glycosides or polyglycosides; saturated or unsaturated C8-C30 monoesters and polyesters of sucrose; polyoxyethylenated or non-polyoxyethylenated, saturated or unsaturated C8-C30 monoesters and polyesters of glycerol; polyoxyethylenated or non-polyoxyethylenated, saturated or unsaturated C8-C30 monoethers or polyethers of glycerol; and mixtures thereof.
1) As poly(oxyethylenated) saturated or unsaturated C8-C30 monoesters or polyesters, use is preferably made of those with a number of ethylene oxide (EO) units ranging from 2 to 200. Mention may be made, for example, of (INCI name) PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate and PEG-40 hydrogenated castor oil.
2) As saturated or unsaturated poly(oxyethylenated) C8-C30 alcohols, use is preferably made of those with a number of ethylene oxide (EO) units ranging from 3 to 15. Mention may be made, for example, of laureth-3, laureth-4, laureth-7, ceteth-5, ceteth-7, oleth-5, oleth-7, oleth-10, oleth-12, steareth-6, ceteareth-7, ceteareth-10, pareth-3, C12-15 pareth-3, C12-13 pareth-4, trideceth-3, trideceth-4, trideceth-5, trideceth-6, trideceth-7 and trideceth-10, and mixtures thereof.
3) Optionally polyoxyethylenated, saturated or unsaturated, C8-C30 monesters or polyesters of sorbitan that are preferably used are those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).
4) Optionally polyoxyethylenated, saturated or unsaturated, C8-C30 mono- or polyethers of sorbitan that are preferably used are those with a number of ethylene oxide (EO) units ranging from 0 to 100.
5) Alkyl or polyalkyl glucosides or polyglucosides that are preferably used are those containing an alkyl group comprising from 6 to 30 carbon atoms and preferably from 6 to 18 or even from 8 to 16 carbon atoms, and containing a glucoside group preferably comprising from 1 to 5 and especially 1, 2 or 3 glucoside units. The alkylpolyglucosides may be chosen, for example, from decylglucoside (alkyl-C9/C11-polyglucoside (1.4)); caprylyl/capryl glucoside; laurylglucoside; cocoglucoside; caprylylglucoside; and mixtures thereof.
6) Examples of C8-C30 mono- or polyesters of sucrose that may be mentioned are CRODESTA™ F150 emulsifier, sucrose monolaurate sold under the name CRODESTA™ SL 40 emulsifier, and the products sold by Ryoto Sugar Ester, for instance sucrose palmitate sold under the reference Ryoto Sugar Ester P1670, Ryoto Sugar Ester LWA 1695 or Ryoto Sugar Ester 01570. Sucrose monooleate, monomyristate and monostearate are also suitable for use.
7) Optionally polyoxyethylenated, saturated or unsaturated, mono- or polyesters of glycerol that are preferably used are those with a number of ethylene oxide (EO) units ranging from 0 to 100 and a number of glycerol units ranging from 1 to 30. Mention may be made, for example, of hexaglyceryl monolaurate, PEG-30 glyceryl stearate, polyglyceryl-2 laurate, polyglyceryl-10 laurate, polyglyceryl-10 stearate, polyglyceryl-10 oleate, PEG-7 glyceryl cocoate and PEG-20 glyceryl isostearate.
8) Optionally polyoxyethylenated mono- or polyethers of glycerol that are preferably used are those with at least one group comprising from 8 to 30 carbon atoms, preferably alkyl, and with a number of ethylene oxide (EO) units ranging from 0 to 100 and a number of glycerol units ranging from 1 to 30.

Preferably, the emulsifier is chosen from poly(oxyethylenated) saturated or unsaturated, mono- or polyesters of glycerol, such as PEG-30 glyceryl stearate.

The emulsifier may be present in a total amount of at least 0.1%, or at least 0.25%, up to no more than 0.5%, no more than 1%, or no more than 2%. In some embodiments, the composition may be substantially free of an emulsifier. In some embodiments, the composition may be free of an emulsifier.

### Rheology Modifier

The composition may include a rheology modifier, such as a lipophilic thickening agent.

The total amount of the rheology modifier may be at least 1%, at least 2%, at least 3%, at least 4%, or at least 5% up to no more than 6%, no more than 7%, no more than 8%, no more than 9%, or no more than 10% by weight of the composition.

### Lipophilic Thickening Agent

In some embodiments, the composition may include a lipophilic thickening agent. The lipophilic thickening agent preferably consists of a modified clay.

Clays are silicates containing a cation that may be chosen from calcium, magnesium, aluminum, sodium, potassium and lithium cations, and mixtures thereof. Examples of such material include, but are not limited to clays of the smectite family, and also of the vermiculite, stevensite and chlorite families. These clays can be of natural or synthetic origin.

Mention may particularly be made of smectites, such as saponites, hectorites, montmorillonites, bentonites or beidellite and in particular synthetic hectorites (also known as laponites), such as the products sold by Rockwood Additives Limited (such products are sodium magnesium silicates and in particular sodium lithium magnesium silicates); bentonites; magnesium aluminum silicates, which are in particular hydrated, or also calcium silicates and in particular that in synthetic form.

In some instances, organophilic clays are preferred, more particularly modified clays, such as montmorillonite, bentonite, hectorite, attapulgite and sepiolite, and mixtures thereof. The clay is preferably an optionally modified bentonite or an optionally modified hectorite. Clays may be modified with a chemical compound chosen from quaternary amines, tertiary amines, amine acetates, imidazolines, amine soaps, fatty sulfates, alkylarylsulfonates and amine oxides, and mixtures thereof.

Mention may be made of hectorites modified with a quaternary amine, more specifically with a C10 to C22 fatty acid ammonium halide, such as a chloride, such as hectorite modified with distearyldimethylammonium chloride (CTFA name: Disteardimonium hectorite), for instance the product sold under the BENTONE® hectorite group of products by the company Elementis, or stearalkonium hectorites.

Mention may also be made of quaternium-18 bentonites; stearalkonium bentonites; or quaternium-18/benzalkonium bentonites.

In some embodiments, two or more rheology modifiers are used. In some embodiments, a single rheology modifier is used. In some embodiments, only lipophilic rheology modifiers are utilized. In some embodiments, only modified clays are utilized. In a preferred embodiment, only disteardimonium hectorite is utilized.

### Film Forming Polymer

The composition may include a film-forming polymer. The film-forming polymer may be water-soluble or a polymer soluble in an oil phase. Non-limiting examples of film-forming polymers include keratin derivatives, such as keratin hydrolysates and sulphonic keratins; anionic, cationic, amphoteric or nonionic derivatives of chitin or chitosan; cellulose derivatives such as hydroxyethylcellulose, hydropropylcellulose, methylcellulose, ethylhydroxyethylcellulose, carboxymethylcellulose, and quaternized derivatives of cellulose; acrylic polymers or copolymers, such as polyacrylates or polymethacrylates (including homo- and co-polymers thereof), such as sodium polymethacrylate; polyvinylpyrrolidones (PVP) and vinyl copolymers, such as allyl stearate/VA copolymer, and VP/eicosene copolymer, methyl vinyl ether-maleic anhydride copolymers, or vinyl acetate-crotonic acid copolymer; polyester, such as adipic acid/diethylene glycol/glycerin crosspolymer; water-dispersible anionic polyesteramide and/or polyester polymers comprising monomers bearing a functional group —SO₃M, in which M represents a hydrogen atom, an ammonium ion NH₄⁺ or a metal ion, such as, for example, an Na⁺, Mg²⁺, Ca²⁺, Cu²⁺, Fe²⁺ or Fe³⁺ ion. Polyurethane polymers, especially anionic, cationic, nonionic or amphoteric polyurethanes, acrylic polyurethanes, polyvinylpyrrolidone polyurethanes, polyester polyurethanes, polyether polyurethanes, polyureas, polyurea/polyurethanes, and mixtures thereof; and polymers of natural origin, modified if desired, such as alginates and carragheenates; glycoaminoglycans, hyaluronic acid and its derivatives; shellac, sandarac gum, dammars, elemis and copals, may also be useful. In an embodiment of the present invention the film-forming polymer is sodium polymethacrylate. The film forming polymers may include vinyl ester homopolymers containing C₈-C₃₀ alkyl groups, such as polyvinyl laurate. The film forming polymers may include polyesters obtained by condensation of an unsaturated fatty acid dimer and/or trimer and of diol, such as. dilinoleic acid/butanediol copolymer). The film forming polymer may include lipophilic polyamide polymers, such as polyamides branched with pendent fatty chains and/or terminal fatty chains containing from 12 to 120 carbon atoms and in particular from 12 to 68 carbon atoms, the terminal fatty chains being bonded to the polyamide backbone via ester groups, such as ethylenediamine/stearyl dimer dilinoleate copolymer.

The total amount of the film forming polymer may be at least 2%, at least 4%, at least 6%, at least 8%, at least 10%, at least 11%, at least 12%, at least 13%, at least 14%, or at least 15% up to no more than 16%, no more than 17%, no more than 18%, no more than 19%, or no more than 20% by weight of the composition, including all ranges and subranges there between.

In some embodiments, the composition may be substantially free of film forming polymer. In some embodiments, the composition may be free of film forming polymer.

### Preservative

The composition may include a preservative. In some embodiments, a preservative may be present in the composition in a total amount of 0-2% by weight of the composition, such as 0-1% by weight of the composition. In some embodiments, the composition may be substantially free of a preservative. In some embodiments, the composition may be free of a preservative.

Non-limiting examples of preservatives include, e.g., ethanol, phenoxyethanol, benzyl alcohol, salicylic acid, sodium benzoate, caprylyl glycol, methyl paraben, propyl paraben, ethylhexylglycerin, 1,2-propanediol or a mixture thereof.

In some embodiments, the composition may include the plurality of waxes, volatile solvent, a rheology modifier, a colorant, a film forming polymer, an emulsifier, and an antioxidant, and may be substantially free, or free, of all other materials.

In some embodiments, excluding any pigment and any rheology modifier, the mascara composition may be substantially free, or free, of particles having a diameter greater than about 5 μm. This may include macroparticles or particulate material included to provide some benefit aside from preservation, opacity, coating smoothness, color, transfer resistance, rheological modification, and/or lash separation, etc.

The mascara composition may be substantially free of fibers. The mascara composition may be free of fibers.

In various aspects, a method for providing improved mascara aesthetics may be provided. The method may include applying a mascara composition as disclosed herein to one or more lashes. The method may include allowing the mascara composition to remain on the one or more lashes for a period of time. The period of time may be, e.g., from 1 hour to 48 hours.

### Example

The following example formulations were produced using conventional manufacturing techniques for mascaras, generally melting the waxes at an elevated temperature, then mixing in other oil-soluble materials and pigments, mixing water and water-soluble materials separately then combining oil- and water-phases to form an emulsion.

TABLE 1

| Material | Ex 1 | Ex 2 | Ex 3 | Ex 4 | Ex 5 | Ex 6 |
|---|---|---|---|---|---|---|
| Sunflower Seed Wax | 8-10 | 8-10 | 7-8 | 7-8 | 4.5-6.5 | 4.5-6.5 |
| Behenates | 4-5 | 5.5-9 | 3-6 | 6.5-10 | 4-6.5 | 7-9 |
| Tribehenin | 2-3 | 2.5-6 | 1-4 | 4-8 | 2-4.5 | 5-6 |
| Behenyl Behenate | 1.5-2.5 | 1.5-3.5 | 1-2.5 | 2.5-5 | 1.5-3.5 | 1.5-3.5 |

| | |
|---|---|
| Other Waxes | ~4% |
| Volatile Solvents | q.s. |
| Water | ~4% |
| Other Non-Oil | ~5% |
| Oil | q.s. |
| Pigment | 3-5% |
| Emulsifier | <1% |
| Film Forming Polymer | 14-16% |
| Rheology Modifier | 4-6% |
| Antioxidant | <1% |

The various formulas were then applied to fake lashes, which were evaluated via image analysis after images were captured, for lash thickness after 14 strokes (thickness being measured in pixels), and number of grains after 14 strokes. Each “grain” generally refers to a glob or irregularly shaped mascara deposit separate from the smooth even mascara deposit (e.g., less detected grains indicate smoother mascara depositions). These were compared to four conventional mascara formulations. Evaluation from a subset of the example and conventional compositions can be found in Table 2

TABLE 2
(Evaluation Data From Subset Of Tested Compositions)

| | Sunflower | Behenyl | | | |
|---|---|---|---|---|---|
| | Seed Wax | Behenate | Tribehenin | Thickness | Grains |
| ID | (w/w %) | (w/w %) | (w/w %) | (pixels) | (#) |
| Test 1 (Ex. 5) | 6.25 | 1.75 | 2.5 | 52.48 | 53.40 |
| Test 2 (Ex. 5) | 5 | 2.5 | 4 | 45.89 | 43.67 |
| Test 3 (Ex. 5) | 6.25 | 3.25 | 2.5 | 44.56 | 48.44 |
| Test 4 (Ex. 6) | 6.25 | 1.75 | 5.5 | 48.43 | 69.57 |
| Test 5 (Ex. 6) | 6.25 | 3.25 | 5.5 | 47.02 | 79.78 |
| Test 6 (Ex. 3) | 7.5 | 1 | 4 | 45.61 | 56.67 |
| Test 7 (Ex. 3) | 7.5 | 2.5 | 1 | 45.83 | 43.78 |
| Test 8 (Ex. 4) | 7.5 | 2.5 | 7 | 39.06 | 85.89 |
| Test 9 (Ex. 4) | 7.5 | 2.5 | 4 | 46.39 | 61.89 |
| Test 10 (Ex. 4) | 7.5 | 4 | 4 | 44.62 | 74.33 |
| Test 11 (Ex. 1) | 8.75 | 1.75 | 2.5 | 49.73 | 57.56 |
| Test 12 (Ex. 2) | 8.75 | 1.75 | 5.5 | 43.12 | 82.78 |
| Test 13 (Ex. 2) | 10 | 2 | 6 | 44.89 | 80.89 |
| Test 14 (Ex. 2) | 10 | 2.5 | 4 | 43.53 | 92.00 |
| Test 15 (Ex. 2) | 8.75 | 3.25 | 5.5 | 41.04 | 111.56 |
| Test 16 (Ex. 2) | 8.75 | 3.25 | 2.5 | 45.34 | 78.11 |
| Conventional 1 | 0 | 0 | 0 | 29.65 | 371.11 |
| Conventional 2 | 0 | 0 | 0 | 40.88 | 176.22 |
| Conventional 3 | 0 | 0 | 0 | 50.61 | 165.00 |
| Conventional 4 | 0 | 0 | 0 | 36.42 | 129.50 |

In Table 2, for thickness, all example formulations had greater thicknesses than two of the conventional mascaras. One exemplary formula, Test Formula 1, had a thickness greater than all others.

For number of grains, all four conventional mascaras had more than 125 grains after 14 strokes, with one conventional mascara (Conventional 1) having over 350 grains. Surprisingly, all example formulations (including Test 1-16) improved over conventional mascaras, with the worst-performing example having almost 15% fewer grains than the best-performing conventional mascara (compare, e.g., Test 15 vs. Conventional 4). Most test formulations had fewer than 90 grains after 14 strokes (including all formulations falling into Exs. 1 and 3-5 from Table 1). All formulations falling into Ex. 1, 3, and 5 from Table 1 had less than 60 grains after 14 strokes.

What is claimed is:

1. A mascara composition, comprising:
a plurality of waxes including sunflower seed wax and a plurality of behenates including tribehenin and behenyl behenate; and
wherein the mascara composition comprises no more than 10% water by weight of the mascara composition.

2. The mascara composition of claim 1, wherein the mascara composition includes 2%-7% water by weight of the mascara composition.

3. The mascara composition of claim 1, wherein the plurality of waxes includes the sunflower seed wax, the tribehenin, the behenyl behenate, and at least one additional wax.

4. The mascara composition of claim 1, wherein the at least one additional wax has a melting temperature at or below 60° C.

5. The mascara composition of claim 1, wherein the at least one additional wax has a melting temperature above 60° C.

6. The mascara composition of claim 1, wherein the plurality of waxes are present in a total amount of 5% to 35% by weight of the mascara composition.

7. The mascara composition of claim 1, wherein the sunflower seed wax, the tribehenin, and the behenyl behenate are present in a total amount of 5% to 20% by weight of the composition.

8. The mascara composition of claim 1, further comprising a colorant.

9. The mascara composition of claim 8, wherein the colorant is a pigment.

10. The mascara composition of claim 1, further comprising a volatile solvent.

11. The mascara composition of claim 1, wherein the mascara composition is substantially free of a non-volatile solvent.

12. A method for providing improved mascara aesthetics, comprising:
applying a mascara composition of claim 1 to one or more lashes; and
allowing the mascara composition to remain on the one or more lashes for a period of time, the period of time being from 1 hour to 48 hours.
